(19) United States
(12) Patent Application Publication
(10) Pub. No.: US 2009/0136976 A1
(43) Pub. Date: May 28, 2009

(54) Luminescence-based composition

(75) Inventors: Hsiao Chung Tsai, Changhua County (TW); Su-Jan Lee, Taipei City (TW); Tzu-I Wu, Changhua (TW); Mei-Fang Su, Hsinchu County (TW); Wen-Pin Hsieh, Miaoli County (TW)

Correspondence Address: BIRCH STEWART KOLASCH & BIRCH, PO BOX 747, FALLS CHURCH, VA, 22040-0747, US

(73) Assignee: INDUSTRIAL TECHNOLOGY RESEARCH INSTITUTE

(21) Appl. No.: 11/882,439

(22) Filed: Aug. 1, 2007

(30) Foreign Application Priority Data
Aug. 2, 2006 (TW) 95128283

(51) Int. Cl. C12Q 1/48; C12Q 1/28; C12Q 1/32

(52) U.S. Cl. 435/15; 435/28; 435/26

(57) ABSTRACT

Luminescence-based compositions for measurement of aspartate aminotranserase, alanine aminotransferase, total-bilirubin, creatinine phosphokinase, or lactate dehydrogenase, wherein the chemiluminescence-based composition comprises 0.01-100 mM of luminol, 0.001-1000 U/mL of horseradish peroxidase (HRP), 0-10% of Triton X-100, 0-100 mM PIP, and 5˜500 mM of buffer at pH 6˜9, and the luminescence-base composition measures Aspartate aminotransferase (AST), alanine aminotransferase (ALT), total bilirubin, or lactate dehydrogenase (LDH).

### BACKGROUND OF THE INVENTION

1. Field of the Invention

The invention relates to a luminescence-based composition and device using the same, and in particular relates to luminescence-based compositions for Aspartate aminotransferase (AST), Alanine aminotransferase (ALT), total-bilirubin, creatine phosphokinase (CPK) and lactate dehydrogenase (LDH).

2. Description of the Related Art

Biochemical analysis of small molecules is a routine procedure in health examination. Based on the analysis, physiological functions such as kidney, liver, or cardiovascular of a patient can be accessed by a physician. Present analysis is mainly based on absorbance or fluorescence which requires a specific light source and is not suitable for home use. Luminescence analysis is highly sensitive and relatively simple in design, and more particularly, most physiological markers or metabolites can be detected by luminescence analysis. Luminescence analysis can be, therefore, used in the development of fast analysis platform, or in the combination of optical sensors and micro-electro-mechanical system (MEMS) to design a portable physiological detector for personal health management.

Current conventional luminescence-based physiological detectors require large amount of samples and cannot be easily manipulated by non-professionals. In addition, the difficulties of serum separation, matrix interference, sensitivity, reproducibility, and simplified machinery design remain to be solved.

Luminescence assay provides sensitivity of hundred or thousand times that of spectroscopic or colorimetric assays and is relatively simple in manipulation. In particular, most physiological markers or metabolites can be measured by luminescence assay. Luminescence can, therefore, be used in the development of fast analysis platform. Luminescent emission is produced when an electron falls from an excited state induced by chemical or biological reaction to a ground state. Luminescent emission can be classified as chemiluminescence and bioluminescence.

Chemiluminescence utilizes compounds such as luminol, 1,2-dioxetane, acridinium esters, and oxalate esters, or their derivatives, of which luminol is the most common. The emission mechanism of luminol is the oxidation in the presence of peroxidase, usually hydrogen peroxide, with an emission length of 450 nm. The reaction can be catalyzed by enzymes such as horseradish peroxidase, micro-peroxidase, catalase, or other substances such as hemoglobin, cytochrome c, Fe(III), and other metal complexes. The emission can be amplified by enhancers such as phenols, naphthols, and amines to elevate sensitivity. Bioluminescence includes firefly luciferase, bacteria luciferase, and aequorin. Among these, luciferin-luciferase derived from firefly and marine bacteria are well-known, having emission length of 580 nm and 490 nm respectively. Accordingly, chemiluminescence analysis is applied in analysis related to oxidation-reduction reaction, and bioluminescence analysis is applied in analysis related to ATP or NAD(P) reaction. One detector is adequate for various reactions since the emission is in the range of visible light. In addition, these reactions are the most important mechanism for various enzyme-substrate reactions and can be applied in a wide field. Related application has been reported, for example, Rauch et al disclosed a chemiluminescent assay using flow injection analysis system with luminol for the detection of choline or phospholipase D; Michel et al. disclosed a three-enzyme detection system using bacteria luciferase for the detection of D-sorbitol with sensitivity of 50 nM in 4-6 min; Eu et al disclosed a firefly luciferase system with ATP competition for the detection of galactose.

In addition to having high sensitivity, luminescence analysis system does not require excitation light source, filter, or electrodes since it only detects photons. Moreover, background interference will not occur since no fluorescence is emitted. Luminescence analysis has wide dynamic range of up to 5 orders, significantly reducing the complexity of sample pretreatment. The analysis is appropriate for quick detection since the emission is completed in a few seconds. Current luminometers adopt photomultiplier tubes (PMT) or avalanche photodiodes (APD) as the detector and are equipped with signal processing system and sample holding device, thus being relatively simple and suitable for miniaturization to achieve portability.

U.S. Pat. No. 4,286,057 discloses a method for the determination of creatine kinase by the reaction of creatine phosphate with adenosinediposphate with the formation of adenosine triphosphate, transformation of the latter with luciferin and oxygen in the presence of luciferase and diadenosine pentaphosphate with the formation of oxyluciferin and adenosine monophosphate, and measurement of the light emitted thereby.

U.S. Pat. No. 4,080,265 discloses a method for detecting the creatine phosphokinase, comprising providing a test fluid; depositing the test fluid on a porous carrier; drying the fluid to provide a dry test specimen stable; introducing the test specimen into a test solution, and determining the occurrence of a measurable optical change.

U.S. Pat. No. 5,817,467 discloses a reagent for quantitatively determining creatine kinase, which comprises substituted or unsubstituted phosphine, a sulfhydryl-containing compound, and a reaction substrate for creatine kinase.

U.S. Pat. No. 5,306,621 discloses an enhanced chemiluminescent assay, in which a dihydrophthalazinedione, a perosidase, and an osxidant are co-reacted in the present of an enhancer.

U.S. Pat. No. 6,919,463 discloses the compounds, and the compounds can be used for detection in assays for peroxide or peroxide-producing enzymes and in assays employing enzyme-labeled specific binding pairs.

However, in the conventional methods, the pretreatment of the sample is necessary, and the analysis time or the reaction volume is large. To improve the sensitivity and the analysis rate of the detection, a detection composition and a detection method are needed.

### BRIEF SUMMARY OF INVENTION

Accordingly, luminescence-based compositions for the measurement of Aspartate aminotransferase (AST), Alanine aminotransferase (ALT), total-bilirubin, lactate dehydrogenase (LDH) or creatine phosphokinase (CPK) are provided.

An embodiment of the luminescence-based composition for the measurement of Aspartate aminotransferase (AST) comprises of 5-100 mM asparate at pH 6.5, 1-500 mM of 2-oxoglutarate, 0.1-100 U/mL of oxaloacetate decarboxylase, 0.1 μM-1 mM of FAD, 0.1-100 mM of TPP, 1 μM-20 mM of MgSO₄, 0.1-100 U/mL of pyruvate oxidase, 0.01-100 mM of luminol, 0.001-1000 U/mL of horseradish peroxidase (HRP), 0-10% of Triton X-100, 0-100 mM of PIP, and 5-500 mM of buffer at pH 6-9.

An embodiment of the luminescence-based composition for the measurement of Alanine aminotransferase (ALT) comprises of 5-500 mM L-alanine at pH 6.5, 5-500 mM of 2-oxoglutarate, 0.1-50 μM of FAD, 0.1-20 mM of TPP, 1 μM-20 mM of MgSO₄, 0.1-50 U/mL of pyruvate oxidase, 0.01-100 mM of luminol, 0.001-1000 U/mL of horseradish peroxidase (HRP), 0-10% of Triton X-100, 0-100 mM of PIP, and 5-500 mM of buffer at pH 6-9.

An embodiment of the luminescence-based composition for the measurement of total-bilirubin comprises of 1-100 U/mL bilirubin oxidase, 0.01-20 mM EDTA, 0.01-100 mM of luminol, 0.001-1000 U/mL of horseradish peroxidase (HRP), 0-10% of Triton X-100, 0-100 mM of PIP, and 5-500 mM of buffer at pH 6-9.

An embodiment of the luminescence-based composition for the measurement of lactate dehydrogenase (LDH) comprises of 5-500 mM glycine, 0.1-100 mM of β-NADH reduced form, 0.1-100 mM of sodium pyruvate, 0.01-1000 U/mL of the lactate oxidase, 0.01-50 mM of DTT (1,4-dithiothreitol), 0.01-100 mM of luminol, 0.001-1000 U/mL of horseradish peroxidase (HRP), 0-1% of BSA, 0-10% of Triton X-100, 0-100 mM of PIP, and 5-500 mM of buffer at pH 6-9.

An embodiment of the luminescence-based composition for the measurement of creatine phosphokinase (CPK) comprises of 0.01-50 mM creatine phosphate, 1×10⁻⁶-5×10⁻² mg/mL of firefly luciferase, 0.1-5000 μM of luciferin, 1 μM-20 mM of MgSO₄, 0.1-20 mM of ADP, 0-1% of BSA, 0-50 mM of DTT (1,4-dithiothreitol), and 1-1000 mM of buffer at pH 6-9.

The invention further provides a method of measuring an analyte, comprising: providing a composition comprising 0.01-100 mM of luminol, 0.001-1000 U/mL of horseradish peroxidase (HRP), 0-10% of Triton X-100, 0-100 mM PIP, 5-500 mM of buffer at pH 6-9, and a additional mixture; providing a biological sample comprising aspartate aminotransferase (AST), alanine aminotransferase (ALT), total bilirubin, or lactate dehydrogenase (LDH) form a subject; mixing the composition and the biological sample, wherein the analyte is aspartate aminotransferase (AST), alanine aminotransferase (ALT), total bilirubin, or lactate dehydrogenase (LDH) form a subject, provided that when the analyte is aspartate aminotransferase (AST), the composition further comprises 5-100 mM of asparate at pH 6.5, 1-500 mM of 2-oxoglutarate, 0.1-100 U/mL of oxaloacetate decarboxylase, 0.1 μM-1 mM of FAD, 0.1-100 mM of TPP, and 0.1-100 U/mL of pyruvate oxidase; when the analyte is alanine aminotransferase (ALT), the composition further comprises 5-500 mM of L-alanine at pH 6.5, 5-500 mM of 2-oxoglutarate, 0.1-50M of FAD, 0.1-20 mM of TPP, 0.1-50 U/mL of pyruvate oxidase; when the analyte is total bilirubin, the composition further comprises 1-100 U/mL of bilirubin oxidase, and 0.01-20 mM EDTA; and when the analyte is lactate dehydrogenase (LDH), the composition further comprises 0.1-100 mM of β-NADH reduced form, 0.1-100 mM of sodium pyruvate, 0.01-1000 U/mL of the lactate oxidase, and 0.01-50 mM of DTT (1,4-dithiothreitol).

The invention further provides a method of measuring the activity of creatine phosphokinase (CPK), comprising: providing a composition comprising 0.01-50 mM of creatine phosphate, 1×10⁻⁶-5×10⁻² mg/mL of firefly luciferase, 0.1-5000 μM of luciferin, 1 μM-20 mM of MgSO₄, 0.1-20 mM of ADP, 0-1% of BSA, 0-50 mM of DTT (1,4-dithiothreitol), and 1-1000 mM of buffer at pH 6-9; providing a biological sample comprising creatine phosphokinase (CPK), and mixing the composition and the biological sample.

A detailed description is given in the following embodiments with reference to the accompanying drawings.

### BRIEF DESCRIPTION OF DRAWINGS

The invention can be more fully understood by reading the subsequent detailed description and examples with references made to the accompanying drawings, wherein:

FIG. 1 shows the calibration curve of the measurement of serum aspartate aminotransferase (AST).

FIG. 2 shows the calibration curve of the measurement of serum alanine aminotransferase (ALT).

FIG. 3 shows the calibration curve of the measurement of serum total bilirubin.

FIG. 4 shows the calibration curve of the measurement of serum lactate dehydrogenase (LDH).

FIG. 5 shows the calibration curve of the measurement of serum creatine phosphokinase (CPK).

### DETAILED DESCRIPTION OF INVENTION

The following description is of the best-contemplated mode of carrying out the invention. This description is made for the purpose of illustrating the general principles of the invention and should not be taken in a limiting sense. The scope of the invention is best determined by reference to the appended claims.

Conventional assessment of liver function cannot be accomplished with a single indicator, so examination items mainly comprise liver function test (such as AST, ALT, and total-bilirubin), immunological examination (such as HBV and HCV), alpha-fetoprotein and abdominal Sonar, wherein AST, ALT and bilirubin are most important and popular. The physiological meaning of those markers is explained thereafter. First, AST exists in heart cells, with a small amount in liver cells, and traces in blood. When tissue cells are pathologically altered, the amount of AST in the blood is increased. Second, ALT exists mostly in liver cells, with less in heart muscle cells, and traces in blood. When liver or heart muscle cells necrotize, the amount of ALT is increased, with the increased value thereof representing the extent of damage. Third, bilirubin is formed at the end of the catabolism pathway of erythrocytes. A part of bilirubin is conjugated with albumin in liver, which directly transfers to water soluable bilirubin. The soluable bilirubin is released through the bile duct. Thus, the amount of bilirubin in the blood indicates the function of bilirubin in liver. Finally, LDH is found in liver, heart muscle, kidney and erythrocyte, with increased amount indicating possible liver disease.

Cardiovascular diseases exhibit many symptoms similar to liver dysfunction. The assessment of cardiovascular disease also cannot be relied on only a single indicator. Nowadays, CPK is a popular indicator for cardiovascular disease. CPK is an enzyme catalyzing converting creatine to phosphocreatine, consuming adenosine triphosphate. CPK increases at 4-6 hours after myocardial infarction. At 24 hours after myocardial infarction, the amount of CPK peaks, then return to normal levels after 3 days. LDH can also be used to diagnose myocardial infarction. The advantage of LDH is that the increase in amounts thereof is slower than that of CPK. LDH is increased at 24-72 hours after myocardial infarction, with peak at 2-5 days after myocardial infarction and remained over normal value after 14 days of the onset of AMI.

A small sample volume and fast analysis method for liver and heart function is thus called for. The analysis method further provides indication of physiology function, assisting health management. Present analysis is mainly based on absorbance or fluorescence which requires a specific light source and is not suitable for household or personal applications. Luminescence analysis is highly sensitive and relatively simple in design, and more particularly, most physiological markers or metabolites can be detected by luminescence analysis. Luminescence analysis can be used in the development of fast analysis platform, or in combination of optical sensors and micro-electro-mechanical system to design a portable physiological detector for personal health management.

The invention provides luminescence-based compositions for the measurement of Aspartate aminotransferase (AST), Alanine aminotransferase (ALT), total-bilirubin, creatine phosphokinase (CPK) or lactate dehydrogenase (LDH) with one-step reaction. The compositions can be used in an aqueous solution or lyophilized powder and are the most appropriate formula for the detection of trace analytes in small sample volume with a stable and reliable sensitivity and a wide detection range.

Accordingly, one embodiment of the luminescence-based composition for the measurement of Aspartate aminotransferase (AST) comprises 5-100 mM of asparate at pH 6.5, 1-500 mM of 2-oxoglutarate, 0.1-100 U/mL of oxaloacetate decarboxylase, 0.1 μM-1 mM of FAD, 0.1-100 mM of TPP, 0.1-100 U/mL of pyruvate oxidase, 0.01-100 mM of luminol, 0.001-1000 U/mL of horseradish peroxidase (HRP), 0-10% of Triton X-100, 0-100 mM of PIP, and 5-500 mM of buffer at pH 6-9.

Another embodiment of the luminescence-based composition for the measurement of Aspartate aminotransferase (AST) comprises 10-50 mM of asparate at pH 6.5, 1-100 mM of 2-oxoglutarate, 0.1-50 U/mL of oxaloacetate decarboxylase, 0.1-100 μM of FAD, 0.1-10 mM of TPP, 10-100 U/mL of pyruvate oxidase, 0.01-20 mM of luminol, 0.01-1000 U/mL of horseradish peroxidase (HRP), 0-2% of Triton X-100, 0-20 mM of PIP, and 10-200 mM of buffer at pH 6˜9.

Another embodiment of the luminescence-based composition for the measurement of Aspartate aminotransferase (AST) comprises 20-40 mM of asparate at pH 6.5, 1-10 mM of 2-oxoglutarate, 1-20 U/mL of oxaloacetate decarboxylase, 1-10 μM of FAD, 0.1-1 mM of TPP, 20-50 U/mL of pyruvate oxidase, 0.1-10 mM of luminol, 0.01-500 U/mL of horseradish peroxidase (HRP), 0-1% of Triton X-100, 0-10 mM of PIP, and 25-100 mM of buffer at pH 6˜9.

The buffer used for the luminescence-based composition for the measurement of aspartate aminotransferase (AST) can comprise, but is not limited to, Gly-gly buffer, HEPES, Tris, Bis-Tris, Bis-Tris propane, MOPS, PIPES, phosphate, or borate, preferably Tris buffer at pH 8.0.

One embodiment of the luminescence-based composition for the measurement of Alanine aminotransferase (ALT) comprises 5-500 mM of L-alanine, 5-500 mM of 2-oxoglutarate, 0.1-50 μM of FAD, 0.1-20 mM of TPP, 0.1-50 U/mL of pyruvate oxidase, 0.01-100 mM of luminol, 0.001-1000 U/mL of horseradish peroxidase (HRP), 0-10% of Triton X-100, 0-100 mM of PIP, and 5-500 mM of buffer at pH 6-9.

Another embodiment of the luminescence-based composition for the measurement of Alanine aminotransferase (ALT) comprises 10-250 mM of L-alanine, 10-100 mM of 2-oxoglutarate, 0.1-20 μM of FAD, 0.1-10 mM of TPP, 0.5-10 U/mL of pyruvate oxidase, 0.01-20 mM of luminol, 0.01-1000 U/mL of horseradish peroxidase (HRP), 0-2% of Triton X-100, 0-20 mM of PIP, and 10-200 mM of buffer at pH 6-9.

Another embodiment of the luminescence-based composition for the measurement of Alanine aminotransferase (ALT) comprises 10-100 mM of L-alanine, 10-50 mM of 2-oxoglutarate, 0.1-10 μM of FAD, 0.1-5 mM of TPP, 1-5 U/mL of pyruvate oxidase, 0.1-10 mM of luminol, 0.01-500 U/mL of horseradish peroxidase (HRP), 0-1% of Triton X-100, 0-10 mM of PIP, and 25-100 mM of buffer at pH 6-9.

The buffer used for the luminescence-based composition for the measurement of Alanine aminotransferase (ALT) can comprise, but is not limited to, Gly-gly buffer, HEPES, Tris, Bis-Tris, Bis-Tris propane, MOPS, PIPES, phosphate, or borate, preferably Tris buffer at pH 8.2.

One embodiment of the luminescence-based composition for the measurement of total bilirubin comprises 1-100 U/mL of bilirubin oxidase, 0.01-20 mM EDTA, 0.01-100 mM of luminol, 0.001-1000 U/mL of horseradish peroxidase (HRP), 0-10% of Triton X-100, 0-100 mM of PIP, and 5-500 mM of buffer at pH 6-9.

Another embodiment of the luminescence-based composition for the measurement of total bilirubin comprises 2-50 U/mL of bilirubin oxidase, 0.1-10 mM EDTA, 0.01-20 mM of luminol, 0.01-1000 U/mL of horseradish peroxidase (HRP), 0-2% of Triton X-100, 0-20 mM of PIP, and 10-200 mM of buffer at pH 6-9.

Another embodiment of the luminescence-based composition for the measurement of total bilirubin comprises 20-40 U/mL of bilirubin oxidase, 0.1-5 mM EDTA, 0.1-10 mM of luminol, 0.01-500 U/mL of horseradish peroxidase (HRP), 0-1% of Triton X-100, 0-10 mM of PIP, and 25-100 mM of buffer at pH 6-9.

The buffer used for the luminescence-based composition for the measurement of total bilirubin can be composed of, but is not limited to, Gly-gly buffer, HEPES, Tris, Bis-Tris, Bis-Tris propane, MOPS, PIPES, phosphate, or borate, preferably Tris buffer at pH 8.5.

One embodiment of the luminescence-based composition for the measurement of lactate dehydrogenase (LDH) comprises 0.1-100 mM of β-NADH reduced form, 0.1-100 mM of sodium pyruvate, 0.01-1000 U/mL of the lactate oxidase, 0.01-50 mM of DTT (1,4-dithiothreitol), 0.01-100 mM of luminol, 0.001-1000 U/mL of horseradish peroxidase (HRP), 0-1% BSA and 5-500 mM of buffer at pH 6-9.

Another embodiment of the luminescence-based composition for the measurement of lactate dehydrogenase (LDH) comprises 1-50 mM of β-NADH reduced form, 1-50 mM of sodium pyruvate, 1-500 U/mL of the lactate oxidase, 0.1-10 mM of DTT (1,4-dithiothreitol), 0.01-20 mM of luminol, 0.01-1000 U/mL of horseradish peroxidase (HRP), 0-1% BSA and 10-200 mM of buffer at pH 6-9.

Another embodiment of the luminescence-based composition for the measurement of lactate dehydrogenase (LDH) comprises 5-20 mM of β-NADH reduced form, 5-20 mM of sodium pyruvate, 10-100 U/mL of the lactate oxidase, 0.1-5 mM of DTT (1,4-dithiothreitol), 0.1-10 mM of luminol, 0.01-500 U/mL of horseradish peroxidase (HRP), 0-1% BSA and 25-100 mM of buffer at pH 6-9.

The buffer used for the luminescence-based composition for the measurement of lactate dehydrogenase (LDH) can comprise, but is not limited to, Gly-gly buffer, HEPES, Tris, Bis-Tris, Bis-Tris propane, MOPS, PIPES, phosphate, or borate, preferably Glycine buffer at pH 7.0.

One embodiment of the luminescence-based composition for the measurement of creatine phosphokinase (CPK) comprises 0.01-50 mM of creatine phosphate, 1×10⁻⁶-5×10⁻² mg/mL of firefly luciferase, 0.1-5000 μM of luciferin, 1 μM-20 mM of MgSO₄, 0.1-20 mM of ADP, 0-1% of BSA, 0-50 mM of DTT (1,4-dithiothreitol), and 1-1000 mM of buffer at pH 6-9.

Another embodiment of the luminescence-based composition for the measurement of creatine phosphokinase (CPK) comprises 0.1-10 mM of creatine phosphate, 5×10⁻⁶-1×10⁻² mg/mL of firefly luciferase, 0.1-500 μM of luciferin, 0.1-10 mM of MgSO₄, 0.1-5 mM of ADP, 0-1% of BSA, 0-20 mM of DTT (1,4-dithiothreitol), and 1-500 mM of buffer at pH 6-9.

Another embodiment of the luminescence-based composition for the measurement of creatine phosphokinase (CPK) comprises 0.1-5 mM of creatine phosphate, 5×10⁻⁵-5×10⁻³ mg/mL of firefly luciferase, 1-50 μM of luciferin, 1-10 mM of MgSO₄, 0.1-1 mM of ADP, 0-1% of BSA, 10-20 mM of DTT (1,4-dithiothreitol), and 5-200 mM of buffer at pH 6-9.

The buffer used for the luminescence-based composition for the measurement of creatine phosphokinase (CPK) can comprise, but is not limited to, Gly-gly buffer, HEPES, Tris, Bis-Tris, Bis-Tris propane, MOPS, PIPES, phosphate, or borate, preferably Gly-gly buffer at pH 7.5.

### EXAMPLES

### Procedures

Preparation of the Embodiments of Chemiluminescent Compositions

The master mixture was prepared in accordance with Table 1. Ten or twenty μl of the master mixture was placed into the testing tube. The master mixture was added to the sample and the RLU value was recorded at an appropriate time by a luminometer when the test was performed in solution form. When the test was performed in lyophilized form, sample was added to the testing tube containing the lyophilized master mixture and the RLU value was recorded at an appropriate time by the luminometer.

Lyophilization

The difference between blank and sample containing analytes is determined by the master mixture. The determination of the master mixture was confirmed by the luminometer. Ten or twenty μl of the master mixture was placed into a testing tube and the solution was frozen in liquid nitrogen for 20 sec. The testing tube was placed in a VirTis Advantage lyophilizer for 6 hours in order to lyophilize the master mixture. The testing tube was then stored at 4° C. in dark.

### Example 1

### Preliminary Experiment for the Measurement of Aspartate Aminotransferase (AST)

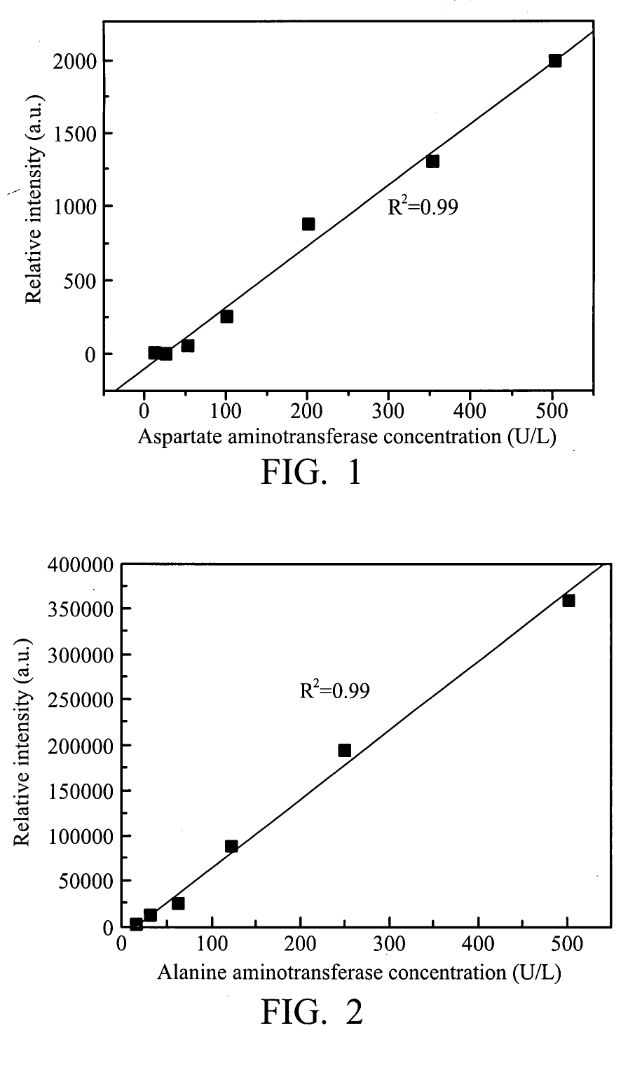

Detection was performed according to the materials and methods disclosed and the compositions listed in Table 1. Nine μL of the master mixture was added to each tube and 1 μL of Aspartate aminotransferase (AST) solution was introduced. The results are shown in FIG. 1.

TABLE 1
chemical compositions for the detection of Aspartate
aminotransferase (AST) in solution form

| Chemicals | Stock solution | | Running conc. | | Amount (μL) |
|---|---|---|---|---|---|
| Asparate | 53.6 | mM | 35.2 | mM | 65.63 |
| Tris buffer, pH 8.0 | 1 | M | 100 | mM | 10 |
| FAD | 10 | mM | 0.02 | mM | 0.2 |
| TPP | 100 | mM | 0.2 | mM | 0.2 |
| MgSO₄ | 1 | M | 10 | mM | 1 |
| Triton X-100 | 0.1% | | 0.001% | | 1 |
| 2-oxoglutarate | 500 | mM | 5 | mM | 1 |
| Luminal | 100 | mM | 1.5 | mM | 1.5 |
| PIP | 50 | mM | 1 | mM | 2 |
| oxaloacetate | 500 | U/mL | 10 | U/mL | 2 |
| decarboxylase | | | | | |
| pyruvate oxidase | 250 | U/mL | 30 | U/mL | 12 |
| horseradish peroxidase | 34.54 | U/mL | 1.2 | U/mL | 3.47 |
| Total | | | | | 100 |

### Example 2

### Preliminary Experiment for the Measurement of Alanine Aminotransferase (ALT)

The detection was performed according to the materials and methods disclosed and the compositions listed in Table 2. Nine μL of the master mixture was added to each tube and 1 μL of Alanine aminotransferase (ALT) solution was introduced. The results are shown in FIG. 2.

TABLE 2
chemical compositions for the detection of Alanine
aminotransferase (ALT) in solution form

| Chemicals | Stock solution | | Running conc. | | Amount (μL) |
|---|---|---|---|---|---|
| PB buffer (pH 6.5) | | | | | 58.1 |
| Tris buffer (pH 8.2) | 1 | M | 200 | mM | 20 |
| L-alanine | 1 | M | 100 | mM | 10 |
| FAD | 10 | mM | 0.02 | mM | 0.2 |
| TPP | 100 | mM | 0.2 | mM | 0.2 |
| Trition X-100 | 0.1% | | 0.001% | | 1 |
| 2-oxoglutrarate | 500 | mM | 20 | mM | 4 |
| luminol | 100 | mM | 1.5 | mM | 1.5 |
| PIP | 50 | mM | 1 | mM | 2 |
| MgSO₄ | 100 | mM | 1 | mM | 1 |
| Pyruvate oxidase | 250 | U/mL | 2.5 | U/mL | 1 |
| horseradish peroxidase | 34.5 | U/mL | 0.345 | U/mL | 1 |

| | |
|---|---|
| Total | 100 |

### Example 3

### Preliminary Experiment for the Measurement of Total-Bilirubin

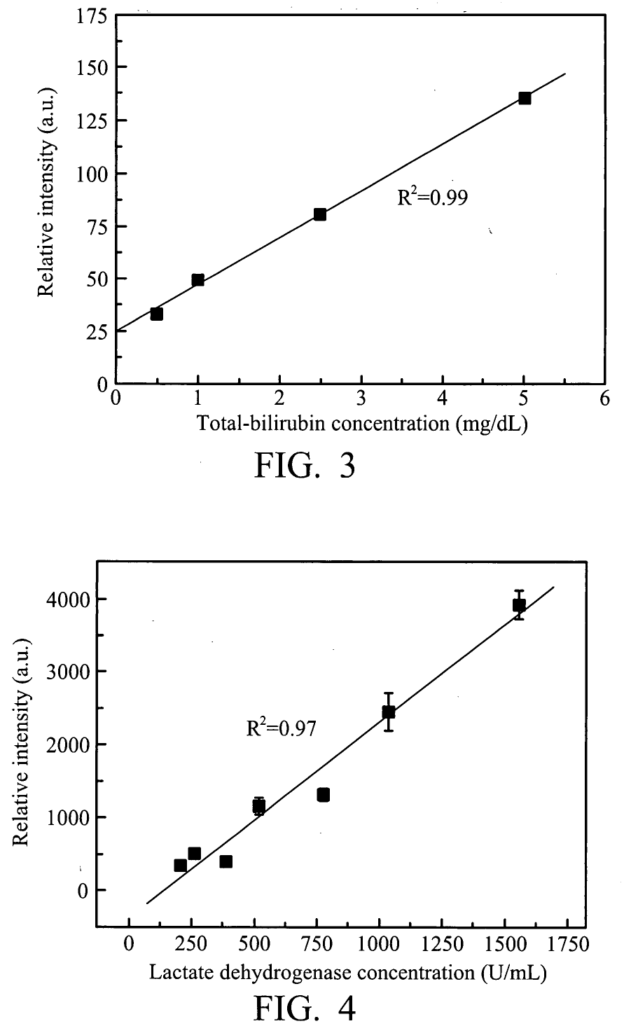

The detection was performed according to the materials and methods disclosed and the compositions listed in table 3. Nine μL of the master mixture was added to each tube and 1 μL of total-bilirubin solution was introduced. The results are shown in FIG. 3.

TABLE 3
chemical compositions for the detection
of total-bilirubin in solution form

| Chemicals | Stock solution | | Running conc. | | Amount (μL) |
|---|---|---|---|---|---|
| H₂O | | | | | 14.6 |
| Tris-HCl, pH 8.5 | 1 | M | 25 | mM | 5 |
| PIP | 50 | mM | 0.5 | mM | 2 |
| Luminol | 100 | mM | 3 | mM | 6 |
| EDTA | 500 | mM | 1 | mM | 0.4 |
| Horseradish peroxidase | lO | U/mL | 0.1 | U/mL | 2 |
| Bilirubin oxidase | 50 | U/mL | 37.5 | U/mL | 150 |

| | |
|---|---|
| Total | 100 |

### Example 4

### Preliminary Experiment for the Measurement of Lactate Dehydrogenase (LDH)

The detection was performed according to the materials and methods disclosed and the compositions listed in table 4. Nine μL of the master mixture was added to each tube and 1 μL of lactate dehydrogenase (LDH) solution was introduced. The results are shown in FIG. 4.

TABLE 4:
chemical compositions for the detection of lactate
dehydrogenase (LDH) in solution form

| Chemicals | Stock solution | | Running conc. | | Amount (μL) |
|---|---|---|---|---|---|
| Glycine, pH 7.0 | 100 | mM | 81 | mM | 80 |
| β-NADH reduce form | 0.51 | M | 15.3 | mM | 3 |
| Sodium pyruvate | 0.55 | M | 16.5 | mM | 3 |
| Luminol | 100 | mM | 5 | mM | 5 |
| DTT | 25 | mM | 1 | mM | 4 |
| BSA | 6.67% | | 0.267% | | 4 |
| Lactate Oxidase | 1000 | U/mL | 50 | U/mL | 0.5 |
| horseradish peroxidase | 3200 | U/mL | 160 | U/mL | 0.5 |
| Total | | | | | 100 |

### Example 5

### Preliminary Experiment for the Measurement of Creatine Phosphokinase (CPK)

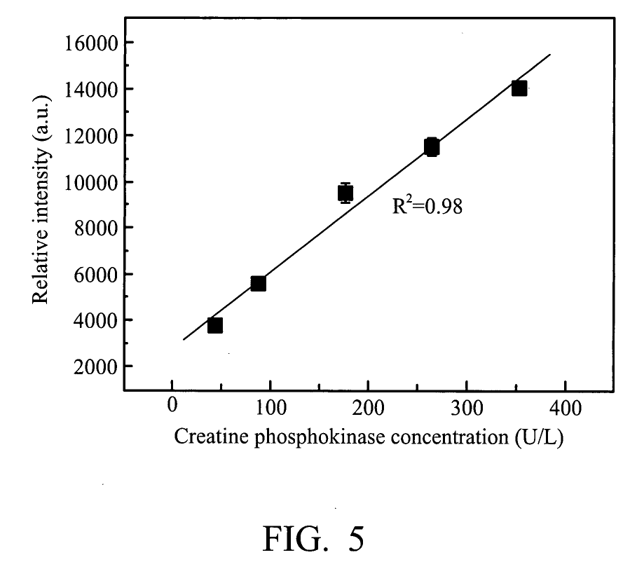

The detection was performed according to the materials and methods disclosed and the compositions listed in table 5. Nine μL of the master mixture was added to each tube and 1 μL of creatine phosphokinase (CPK) solution was introduced. The results are shown in FIG. 5.

TABLE 5
chemical compositions for the detection of creatine
phosphokinase (CPK) in solution form

| Chemicals | Stock solution | | Running conc. | | Amount (μL) |
|---|---|---|---|---|---|
| Gly-gly buffer, pH 7.5 | 100 | mM | 67 | mM | 67 |
| Creatine phosphate | 28.9 | mM | 2.89 | mM | 10 |
| ADP | 22.9 | mM | 0.229 | mM | 1 |
| Luciferin | 1.78 | mM | 17.8 | mM | 1 |
| DTT | 25 | mM | 1 | mM | 4 |
| BSA | 6.67% | | 0.13% | | 2 |
| MgSO4 | 300 | mM | 30 | mM | 10 |
| Firefly luciferase | 0.25 | mg/ml | 0.0125 | mg/ml | 5 |

| | |
|---|---|
| Total | 100 |

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A chemiluminescence-based composition, comprises 0.01-100 mM of luminol, 0.001-1000 U/mL of horseradish peroxidase (HRP), 0-10% of Triton X-100, 0-100 mM PIP, and 5˜500 mM of buffer at pH 6˜9, and the luminescence-base composition measures Aspartate aminotransferase (AST), alanine aminotransferase (ALT), total bilirubin, or lactate dehydrogenase (LDH).

2. The luminescence-based composition as claimed in claim 1, wherein the luminescence-base composition of Aspartate aminotransferase (AST) comprises 5-100 mM of asparate at pH 6.5, 1-500 mM of 2-oxoglutarate, 0.1-100 U/mL of oxaloacetate decarboxylase, 0.1 μM-1 mM of FAD, 0.1-100 mM of TPP, 0.1-100 U/mL of pyruvate oxidase, 0.01-100 mM of luminol, 0.001-1000 U/mL of horseradish peroxidase (HRP), 0-10% of Triton X-100, 0-100 mM of PIP, and 5-500 mM of buffer at pH 6-9.

3. The luminescence-based composition as claimed in claim 2, wherein the buffer is Gly-gly buffer, HEPES, Tris, Bis-Tris, Bis-Tris propane, MOPS, PIPES, phosphate, or borate.

4. The luminescence-based composition as claimed in claim 3, wherein the buffer is Tris buffer at pH 6.5.

5. The luminescence-based composition as claimed in claim 1, wherein the luminescence-base composition of Aspartate aminotransferase (AST) comprises 10-50 mM of asparate at pH 6.5, 1-100 mM of 2-oxoglutarate, 0.1-50 U/mL of oxaloacetate decarboxylase, 0.1-100 μM of FAD, 0.1-10 mM of TPP, 10-100 U/mL of pyruvate oxidase, 0.01-20 mM of luminol, 0.01-1000 U/mL of horseradish peroxidase (HRP), 0-2% of Triton X-100, 0-20 mM of PIP, and 10-200 mM of buffer at pH 6˜9.

6. The luminescence-based composition as claimed in claim 5, wherein the buffer is Gly-gly buffer, HEPES, Tris, Bis-Tris, Bis-Tris propane, MOPS, PIPES, phosphate, or borate.

7. The luminescence-based composition as claimed in claim 6, wherein the buffer is Tris buffer at pH 6.5.

8. The luminescence-based composition as claimed in claim 1, wherein the luminescence-base composition of Aspartate aminotransferase (AST) comprises 20-40 mM of asparate at pH 6.5, 1-10 mM of 2-oxoglutarate, 1-20 U/mL of oxaloacetate decarboxylase, 1-10 μM of FAD, 0.1-1 mM of TPP, 20-50 U/mL of pyruvate oxidase, 0.1-10 mM of luminol, 0.01-500 U/mL of horseradish peroxidase (HRP), 0-1% of Triton X-100, 0-10 mM of PIP, and 25-100 mM of buffer at pH 6˜9.

9. The luminescence-based composition as claimed in claim 8, wherein the buffer is Gly-gly buffer, HEPES, Tris, Bis-Tris, Bis-Tris propane, MOPS, PIPES, phosphate, or borate.

10. The luminescence-based composition as claimed in claim 9, wherein the buffer is Tris buffer at pH 6.5.

11. The luminescence-based composition as claimed in claim 1, wherein the luminescence-base composition of Alanine aminotransferase (ALT) comprises 5-500 mM of L-alanine at pH 6.5, 5-500 mM of 2-oxoglutarate, 0.1-50 μM of FAD, 0.1-20 mM of TPP, 0.1-50 U/mL of pyruvate oxidase, 0.01-100 mM of luminol, 0.001-1000 U/mL of horseradish peroxidase (HRP), 0-10% of Triton X-100, 0-100 mM of PIP, and 5-500 mM of buffer at pH 6-9.

12. The luminescence-based composition as claimed in claim 11, wherein the buffer is Gly-gly buffer, HEPES, Tris, Bis-Tris, Bis-Tris propane, MOPS, PIPES, phosphate, or borate.

13. The luminescence-based composition as claimed in claim 12, wherein the buffer is Tris buffer at pH 8.2.

14. The luminescence-based composition as claimed in claim 1, wherein the luminescence-base composition of alanine aminotransferase (ALT) comprises 10-250 mM of L-alanine at pH 6.5, 10-100 mM of 2-oxoglutarate, 0.1-20 μM of FAD, 0.1-10 mM of TPP, 0.5-10 U/mL of pyruvate oxidase, 0.01-20 mM of luminol, 0.01-1000 U/mL of horseradish peroxidase (HRP), 0-2% of Triton X-100, 0-20 mM of PIP, and 10-200 mM of buffer at pH 6-9.

15. The luminescence-based composition as claimed in claim 14, wherein the buffer is Gly-gly buffer, HEPES, Tris, Bis-Tris, Bis-Tris propane, MOPS, PIPES, phosphate, or borate.

16. The luminescence-based composition as claimed in claim 15, wherein the buffer is Tris buffer at pH 8.2.

17. The luminescence-based composition as claimed in claim 1, wherein the luminescence-base composition of Alanine aminotransferase (ALT) comprises 10-100 mM of L-alanine at pH 6.5, 10-50 mM of 2-oxoglutarate, 1-10 μM of FAD, 0.1-5 mM of TPP, 1-5 U/mL of pyruvate oxidase, 0.1-10 mM of luminol, 0.01-500 U/mL of horseradish peroxidase (HRP), 0-1% of Triton X-100, 0-10 mM of PIP, and 25-100 mM of buffer at pH 6-9.

18. The luminescence-based composition as claimed in claim 17, wherein the buffer is Gly-gly buffer, HEPES, Tris, Bis-Tris, Bis-Tris propane, MOPS, PIPES, phosphate, or borate.

19. The luminescence-based composition as claimed in claim 18, wherein the buffer is Tris buffer at pH 8.2.

20. The luminescence-based composition as claimed in claim 1, wherein the luminescence-base composition of total bilirubin comprises 1-100 U/mL of bilirubin oxidase, 0.01-20 mM EDTA, 0.01-100 mM of luminol, 0.001-1000 U/mL of horseradish peroxidase (HRP), 0-10% of Triton X-100, 0-100 mM of PIP, and 5-500 mM of buffer at pH 6-9.

21. The luminescence-based composition as claimed in claim 20, wherein the buffer is Gly-gly buffer, HEPES, Tris, Bis-Tris, Bis-Tris propane, MOPS, PIPES, phosphate, or borate.

22. The luminescence-based composition as claimed in claim 21, wherein the buffer is Tris buffer at pH 8.5.

23. The luminescence-based composition as claimed in claim 1, wherein the luminescence-base composition of total bilirubin comprises 2-50 U/mL of bilirubin oxidase, 0.1-10 mM EDTA, 0.01-20 mM of luminol, 0.01-1000 U/mL of horseradish peroxidase (HRP), 0-2% of Triton X-100, 0-20 mM of PIP, and 10-200 mM of buffer at pH 6-9.

24. The luminescence-based composition as claimed in claim 23, wherein the buffer is Gly-gly buffer, HEPES, Tris, Bis-Tris, Bis-Tris propane, MOPS, PIPES, phosphate, or borate.

25. The luminescence-based composition as claimed in claim 24, wherein the buffer is Tris buffer at pH 8.5.

26. The luminescence-based composition as claimed in claim 1, wherein the luminescence-base composition of total bilirubin comprises 20-40 U/mL of bilirubin oxidase, 0.1-5 mM EDTA, 0.1-10 mM of luminol, 0.01-500 U/mL of horseradish peroxidase (HRP), 0-1% of Triton X-100, 0-10 mM of PIP, and 25-100 mM of buffer at pH 6-9.

27. The luminescence-based composition as claimed in claim 26, wherein the buffer is Gly-gly buffer, HEPES, Tris, Bis-Tris, Bis-Tris propane, MOPS, PIPES, phosphate, or borate.

28. The luminescence-based composition as claimed in claim 27, wherein the buffer is Tris buffer at pH 8.5.

29. The luminescence-based composition as claimed in claim 1, wherein the luminescence-base composition of lactate dehydrogenase (LDH) comprises 0.1-100 mM of β-NADH reduced form, 0.1-100 mM of sodium pyruvate, 0.01-1000 U/mL of the lactate oxidase, 0.01-50 mM of DTT (1,4-dithiothreitol), 0.01-100 mM of luminol, 0.001-1000 U/mL of horseradish peroxidase (HRP), 0-1% BSA and 5-500 mM of buffer at pH 6-9.

30. The luminescence-based composition as claimed in claim 29, wherein the buffer is Gly-gly buffer, Glycine buffer, HEPES, Tris, Bis-Tris, Bis-Tris propane, MOPS, PIPES, phosphate, or borate.

31. The luminescence-based composition as claimed in claim 30, wherein the buffer is Glycine buffer at pH 7.0.

32. The luminescence-based composition as claimed in claim 1, wherein the luminescence-base composition of lactate dehydrogenase (LDH) comprises 1-50 mM of β-NADH reduced form, 1-50 mM of sodium pyruvate, 1-500 U/mL of the lactate oxidase, 0.1-10 mM of DTT (1,4-dithiothreitol), 0.01-20 mM of luminol, 0.01-1000 U/mL of horseradish peroxidase (HRP), 0-1% BSA and 10-200 mM of buffer at pH 6-9.

33. The luminescence-based composition as claimed in claim 32, wherein the buffer is Gly-gly buffer, Glycine buffer, HEPES, Tris, Bis-Tris, Bis-Tris propane, MOPS, PIPES, phosphate, or borate.

34. The luminescence-based composition as claimed in claim 33, wherein the buffer is Glycine buffer at pH 7.0.

35. The luminescence-based composition as claimed in claim 1, wherein the luminescence-base composition of lactate dehydrogenase (LDH) comprises 5-20 mM of β-NADH reduced form, 5-20 mM of sodium pyruvate, 10-100 U/mL of the lactate oxidase, 0.1-5 mM of DTT (1,4-dithiothreitol), 0.1-10 mM of luminol, 0.01-500 U/mL of horseradish peroxidase (HRP), 0-1% BSA and 25-100 mM of buffer at pH 6-9.

36. The luminescence-based composition as claimed in claim 35, wherein the buffer is Gly-gly buffer, Glycine buffer, HEPES, Tris, Bis-Tris, Bis-Tris propane, MOPS, PIPES, phosphate, or borate.

37. The luminescence-based composition as claimed in claim 36, wherein the buffer is Glycine buffer at pH 7.0.

38. A luminescence-based composition, comprises 1×10⁻⁶-5×10⁻² mg/mL of firefly luciferase, 0.1-5000 μM of luciferin, 1 μM-20 mM of MgSO₄, 0-50 mM of DTT (1,4-dithiothreitol), and 1-1000 mM of buffer at pH 6-9, and the luminescence-base composition measuring creatine phosphokinase (CPK).

39. The luminescence-based composition as claimed in claim 38, wherein the luminescence-base composition of creatine phosphokinase (CPK) comprises 0.01-50 mM of creatine phosphate, 1×10⁻⁶-5×10⁻² mg/mL of firefly luciferase, 0.1-5000 μM of luciferin, 1 μM-20 mM of MgSO₄, 0.1-20 mM of ADP, 0-1% of BSA, 0-50 mM of DTT (1,4-dithiothreitol), and 1-1000 mM of buffer at pH 6-9.

40. The luminescence-based composition as claimed in claim 39, wherein the buffer is Gly-gly buffer, HEPES, Tris, Bis-Tris, Bis-Tris propane, MOPS, PIPES, phosphate, or borate.

41. The luminescence-based composition as claimed in claim 40, wherein the buffer is Gly-gly buffer at pH 7.5.

42. The luminescence-based composition as claimed in claim 38, wherein the luminescence-base composition of creatine phosphokinase (CPK) comprises 0.1-10 mM of creatine phosphate, 5×10⁻⁶-1×10⁻² mg/mL of firefly luciferase, 0.1-500 μM of luciferin, 0.1-10 mM of MgSO₄, 0.1-5 mM of ADP, 0-1% of BSA, 0-20 mM of DTT (1,4-dithiothreitol), and 1-500 mM of buffer at pH 6-9.

43. The luminescence-based composition as claimed in claim 42, wherein the buffer is Gly-gly buffer, HEPES, Tris, Bis-Tris, Bis-Tris propane, MOPS, PIPES, phosphate, or borate.

44. The luminescence-based composition as claimed in claim 43, wherein the buffer is Gly-gly buffer at pH 7.5.

45. The luminescence-based composition as claimed in claim 38, wherein the luminescence-base composition of creatine phosphokinase (CPK) comprises 0.1-5 mM of creatine phosphate, 5×10⁻⁵-5×10⁻³ mg/mL of firefly luciferase, 1-50 μM of luciferin, 1-10 mM of MgSO₄, 0.1-1 mM of ADP, 0-1% of BSA, 10-20 mM of DTT (1,4-dithiothreitol), and 5-200 mM of buffer at pH 6-9.

46. The luminescence-based composition as claimed in claim 45, wherein the buffer is Gly-gly buffer, HEPES, Tris, Bis-Tris, Bis-Tris propane, MOPS, PIPES, phosphate, or borate.

47. The luminescence-based composition as claimed in claim 46, wherein the buffer is Gly-gly buffer at pH 7.5.

48. A method of measuring an analyte, comprising
providing a composition comprising 0.01-100 mM of luminol, 0.001-1000 U/mL of horseradish peroxidase (HRP), 0-10% of Triton X-100, 0-100 mM PIP, 5-500 mM of buffer at pH 6-9, and a additional mixture;
providing a biological sample comprising aspartate aminotransferase (AST), alanine aminotransferase (ALT), total bilirubin, or lactate dehydrogenase (LDH) form a subject;
mixing the composition and the biological sample, wherein the analyte is aspartate aminotransferase (AST), alanine aminotransferase (ALT), total bilirubin, or lactate dehydrogenase (LDH) form a subject, provided that when the analyte is aspartate aminotransferase (AST), the composition further comprises 5-100 mM of asparate at pH 6.5, 1-500 mM of 2-oxoglutarate, 0.1-100 U/mL of oxaloacetate decarboxylase, 0.1 μM-1 mM of FAD, 0.1-100 mM of TPP, and 0.1-100 U/mL of pyruvate oxidase; when the analyte is alanine aminotransferase (ALT), the composition further comprises 5-500 mM of L-alanine at pH 6.5, 5-500 mM of 2-oxoglutarate, 0.1-50 μM of FAD, 0.1-20 mM of TPP, 0.1-50 U/mL of pyruvate oxidase; when the analyte is total bilirubin, the composition further comprises 1-100 U/mL of bilirubin oxidase, and 0.01-20 mM EDTA; and when the analyte is lactate dehydrogenase (LDH), the composition further comprises 0.1-100 mM of β-NADH reduced form, 0.1-100 mM of sodium pyruvate, 0.01-1000 U/mL of the lactate oxidase, and 0.01-50 mM of DTT (1,4-dithiothreitol).

49. The method as claimed in claim 48, wherein the activity of aspartate aminotransferase (AST) is between about 10 U/L-500 U/L.

50. The method as claimed in claim 48, wherein the activity of alanine aminotransferase (ALT) is between about 10 U/L-500 U/L.

51. The method as claimed in claim 48, wherein the concentration of total bilirubin is between about 0.5 mg/dL-5 mg/dL.

52. The method as claimed in claim 48, wherein the activity of lactate dehydrogenase (LDH) is between about 200 U/L-1600 U/L.

53. The method as claimed in claim 48, wherein the volume of the biological sample is less than 10 μl.

54. A method of measuring the activity of creatine phosphokinase (CPK), comprising
providing a composition comprising 0.01-50 mM of creatine phosphate, 1×10⁻⁶-5×10⁻² mg/mL of firefly luciferase, 0.1-5000 μM of luciferin, 1 μM-20 mM of MgSO₄, 0.1-20 mM of ADP, 0-1% of BSA, 0-50 mM of DTT (1,4-dithiothreitol), and 1-1000 mM of buffer at pH 6-9;
providing a biological sample comprising creatine phosphokinase (CPK), and
mixing the composition and the biological sample.

55. The method as claimed in claim 54, wherein the activity of creatine phosphokinase (CPK) is between 40 U/L-350 U/L.

56. The method as claimed in claim 54, wherein the volume of the biological sample is less than 10 μl.
